(19) United States
(12) Patent Application Publication
(10) Pub. No.: US 2023/0366786 A1
(43) Pub. Date: Nov. 16, 2023

(54) HAIR COLLECTION TOOL

(71) Applicant: Aderans Company Limited, Tokyo (JP)

(72) Inventors: Kouhei IGARASHI, Shinjuku-ku, Tokyo (JP); Chie TAKITA, Shinjuku-ku, Tokyo (JP)

(21) Appl. No.: 18/029,250

(22) PCT Filed: Jul. 28, 2022
(86) PCT No.: PCT/JP2022/029116

§ 371 Date: Mar. 29, 2023

(30) Foreign Application Priority Data
Aug. 16, 2021 (JP) 2021-132285

(51) Int. Cl. G01N 1/08

(52) U.S. Cl. CPC G01N 1/08; G01N 33/4833

(57) ABSTRACT

A hair collection tool includes a pedestal portion having a thin plate shape and having a collecting surface; a holding base attached to the collecting surface, and a fixing portion attached to the collecting surface through the holding base or directly. The holding base includes a side surface erected at a position away from an end portion of the pedestal portion by a first distance, and a holding surface extending along the collecting surface in a direction away from the end portion from a position by a second distance above the collecting surface of the side surface. The first distance is longer than a thickness of scissor blades that cut hair, and the second distance is longer than a width of the scissor blades.

### TECHNICAL FIELD

The present invention relates to a hair collection tool used for collecting a hair sample.

### BACKGROUND ART

In recent years, there is an increasing interest in preventative medicine tests that use hair to check the tendency of mineral balance and the like in the body. Among them, a health condition investigation system of testing the accumulation amount in the body of minerals using hair cut at a hair salon has also been proposed (see Patent Literature 1, for example).

### CITATION LIST

### Patent Literature

Patent Literature 1: JP 2021-92968 A

### SUMMARY OF INVENTION

### Technical Problem

In order to appropriately perform such a test, it is important to collect a hair sample including a region near the scalp. However, in the system described in Patent Literature 1, since hair cut at a hair salon is used, a hair sample including a region near the scalp cannot be reliably collected, and there is a possibility that an accurate test using hair cannot be performed. On the other hand, when the user cuts his/her own hair using scissors by himself/herself to collect a hair sample, this becomes a very difficult work, and in this case as well, there is a possibility that an accurate test using hair cannot be performed. In addition, when the hair sample is put in an envelope and mailed, the hair sample may be damaged during the mailing.

Therefore, the present invention has been made in view of the above problems, and an object of the present invention is to provide a hair collection tool that allows anyone to easily collect a hair sample suitable for analysis and to send the collected hair sample to an inspection institution without damaging the hair sample.

A hair collection tool according to one aspect of the present invention includes: a pedestal portion having a thin plate shape and having a collecting surface; a holding base attached to the collecting surface; and a fixing portion attached to the collecting surface through the holding base or directly. The holding base includes a side surface erected at a position away from an end portion of the pedestal portion by a first distance, and a holding surface extending along the collecting surface in a direction away from the end portion from a position by a second distance above the collecting surface of the side surface. The first distance is longer than a thickness of scissor blades that cut hair, and the second distance is longer than a width of the scissor blades. In a state of the end portion being applied to a scalp, a predetermined amount of hair is taken out from a scalp side so that the predetermined amount of hair is placed on the holding surface, hair taken out is fixed by the fixing portion, scissor blades are inserted into a space surrounded by the side surface, the collecting surface, and a scalp to cut hair, and a hair sample is collected.

### Advantageous Effects of Invention

As described above, the present invention can provide a hair collection tool that allows anyone to easily collect a hair sample suitable for analysis and to send the collected hair sample to an inspection institution without damaging the hair sample.

### BRIEF DESCRIPTION OF DRAWINGS

FIG. 1 is a perspective view schematically showing a hair collection tool according to a first embodiment of the present invention.

FIG. 2A is a plan view schematically showing the hair collection tool according to the first embodiment of the present invention.

FIG. 2B is a side view showing a view from arrows A-A in FIG. 2A.

FIG. 2C is a side view showing a view from arrows B-B in FIG. 2A.

FIG. 3A is a diagram schematically showing a position where a hair sample is collected, and a side view showing the back of the head.

FIG. 3B is a diagram schematically showing a state where the hair collection tool is applied to the scalp in order to collect a hair sample using the hair collection tool according to the first embodiment of the present invention.

FIG. 3C is a side view schematically showing a state where the hair is taken out from the scalp so as to be placed on the holding surface in order to collect a hair sample, from the state in FIG. 3B.

FIG. 3D is a plan view of the state shown in FIG. 3C as viewed from the collecting surface side.

FIG. 3E is a side view schematically showing a state where the hair taken out is fixed by the adhesive layer of the fixing portion, from the state shown in FIGS. 3C and 3D.

FIG. 3F is a side view schematically showing a state of cutting hair by inserting scissor blades into a space surrounded by a side surface, a collecting surface, and a scalp, from the state shown in FIG. 3E.

FIG. 4 is a perspective view schematically showing a hair collection tool according to a second embodiment of the present invention.

FIG. 5A is a plan view schematically showing the hair collection tool according to the second embodiment of the present invention.

FIG. 5B is a side view showing a view from arrows C-C in FIG. 5A.

FIG. 5C is a side view showing a view from arrows D-D in FIG. 5A.

FIG. 6 is a side view schematically showing a state of inserting scissor blades into a space surrounded by the side surface, the collecting surface, and the scalp to cut hair in order to collect a hair sample using a hair collection tool according to the second embodiment of the present invention.

FIG. 7A is a perspective view schematically showing a hair collection tool according to a third embodiment of the present invention.

FIG. 7B is a perspective view schematically showing a periphery of a holding base and a fixing portion of the hair collection tool shown in FIG. 7A.

FIG. 8A is a plan view schematically showing the hair collection tool according to the third embodiment of the present invention.

FIG. 8B is a side view showing a view from arrows E-E in FIG. 8A.

FIG. 8C is a side view showing a view from arrows F-F in FIG. 8A.

FIG. 9A is a side view showing a view from arrows G-G in FIG. 8A, and particularly, is a view showing a state in which the fixing portion is opened.

FIG. 9B is a side view showing a view from arrows G-G in FIG. 8A, and particularly, is a view showing a state in which the fixing portion is further rotated in a closing direction from the state shown in FIG. 9A.

FIG. 9C is a side view showing a view from arrows G-G in FIG. 8A, and particularly, is a view showing a state in which the fixing portion is closed.

FIG. 10A is a side view showing a view from arrows G-G in FIG. 8A, and particularly, is a view showing a function of holding hair by a beam portion immediately before the fixing portion is closed.

FIG. 10B is a side view showing a view from arrows G-G in FIG. 8A, and particularly, is a view showing a function of holding hair by a beam portion in a state where the fixing portion is closed.

FIG. 11A is a side view showing a view from arrows J-J in FIG. 8A, and particularly, is a view showing functions of the holding protruding portion and the holding recessed portion, and is a view showing a state in which the fixing portion is opened.

FIG. 11B is a side view showing a view from arrows J-J in FIG. 8A, and particularly, is a view showing functions of the holding protruding portion and the holding recessed portion, and is a view showing a state in which the fixing portion is closed.

FIG. 12A is a side view schematically showing a state where in order to collect a hair sample using the hair collection tool according to the third embodiment of the present invention, the hair collection tool is applied to the scalp, and the hair is taken out from the scalp so as to be placed on a holding surface of a holding base.

FIG. 12B is a side view schematically showing a state where the hair taken out is sandwiched and fixed by the holding surface of the holding base and the fixing surface of the fixing portion, and scissor blades are inserted into a space surrounded by the side surface, the collecting surface, and the scalp to cut the hair, from the state shown in FIG. 12A.

### DESCRIPTION OF EMBODIMENTS

Hereinafter, embodiments for carrying out the present invention will be described with reference to the drawings. It should be noted that the embodiments described below are intended to embody the technical idea of the present invention, and the present invention is not limited to the following embodiments unless otherwise specified.

In each of the drawings, members having the same function may be denoted by the same reference numeral. In consideration of the description of the main points or ease of understanding, the embodiments may be shown separately for convenience, but partial replacement or combination of configurations shown in different embodiments is possible. In the embodiments described below, descriptions of matters common to the above-described embodiment will be omitted, and only different points will be described. In particular, the same action and effect by the same configuration will not be mentioned one by one for each embodiment. The sizes, positional relationships, and the like of the members shown in each drawing may be exaggeratedly shown for clarity of description.

### (Hair Collection Tool According to First Embodiment)

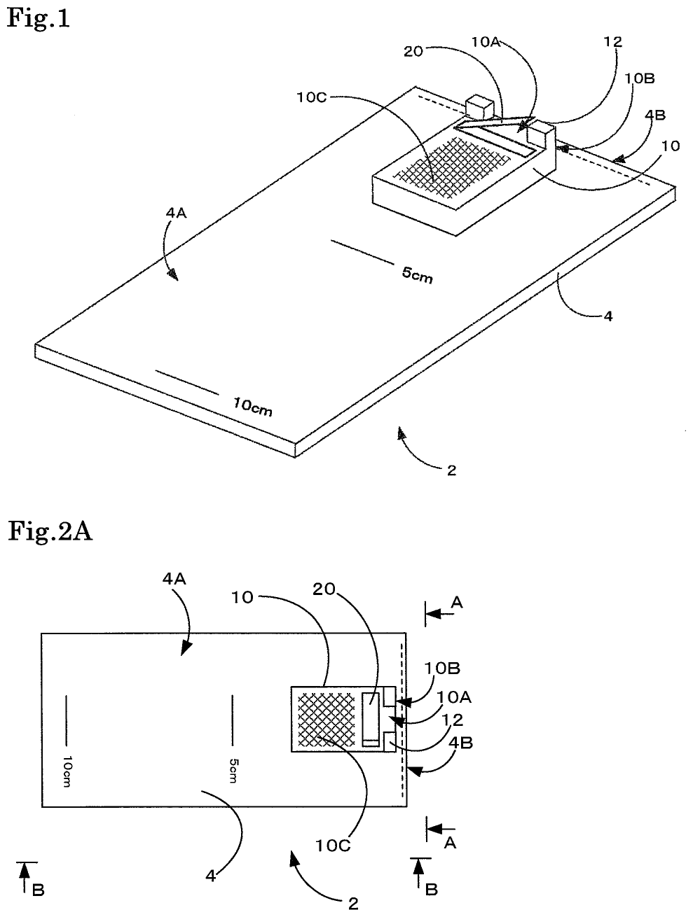

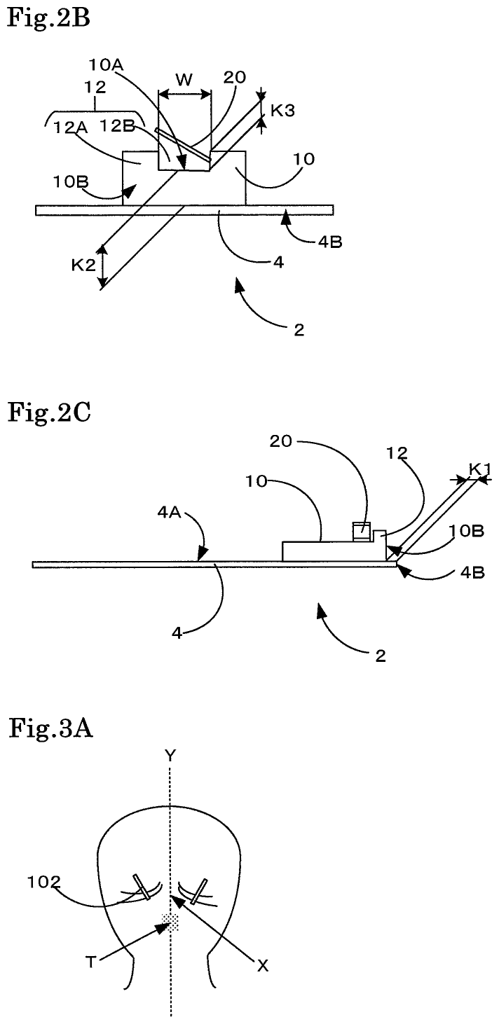

First, a hair collection tool according to a first embodiment of the present invention will be described with reference to FIG. 1 and FIGS. 2A to 2C. FIG. 1 is a perspective view schematically showing a hair collection tool according to the first embodiment of the present invention. FIG. 2A is a plan view schematically showing a hair collection tool according to the first embodiment of the present invention. FIG. 2B is a side view showing a view from arrows A-A in FIG. 2A. FIG. 2C is a side view showing a view from arrows B-B in FIG. 2A.

The hair collection tool 2 according to the present embodiment includes a thin plate-shaped pedestal portion 4 having a collecting surface 4A, a holding base 10 attached to the collecting surface 4A, and a fixing portion 20 attached to an upper surface of the holding base 10. That is, the fixing portion 20 is attached to the collecting surface 4A with interposition of the holding base 10.

The pedestal portion 4 is a thin plate-shaped member made of paper or a resin sheet. The pedestal portion 4 may include a cover member made of resin on a surface of a core material made of paper. For example, even when the end portion 4B of the pedestal portion 4 is applied to the scalp and pressed with a finger from the opposite end portion, it is preferable to have rigidity that does not bend easily. The planar shape of the pedestal portion 4 according to the present embodiment is substantially rectangular. It can be exemplified that the length of the substantially rectangular shape is in a range of 50 mm or more and 150 mm or less, and the width of the substantially rectangular shape is in a range of 30 mm or more and 80 mm or less. However, the planar shape of the pedestal portion 4 is not limited to a substantially rectangular shape, and may have any other planar shape including an elliptical shape.

The holding base 10 includes a side surface 10B erected at a position away from the end portion 4B of the pedestal portion 4 by a distance K1, and a holding surface 10A extending along the collecting surface 4A in a direction away from the end portion 4B from a-position above the collecting surface 4A of the side surface 10B by a distance K2. The distance K1 and the distance K2 can form a space into which scissor blades for cutting hair is inserted as described below. Therefore, the distance K1 is longer than the thickness of the scissor blade for cutting hair, and the distance K2 is longer than the width of the scissor blade. The “thickness and width” of the blade referred to here mean a thickness and a width having large values among the thickness and width of the cutting region of the upper blade and the lower blade of the scissors.

In the present embodiment, the holding surface 10A is disposed substantially parallel to the collecting surface 4A, but the present invention is not limited thereto. For example, there may also be a case where the holding surface 10A is disposed slightly inclined with respect to the collecting surface 4A.

Furthermore, the holding base 10 includes a groove portion 12 in which a recessed portion 12B is disposed between the two protruding portions 12A. The holding surface 10A corresponds to a bottom surface of the groove portion 12. The holding base 10 has a substantially rectangular-parallelepiped shape being substantially flat, and two protruding portions 12A are formed on the side surface 4B side in a protruding manner from the upper surface. Due to this flat shape, the holding base 10 is not bulky even during the mail or the like, and there is little possibility that the holding base 10 is detached from the pedestal portion 4 during the mail. The holding base 10 can be manufactured by integral molding of resin, for example.

In FIG. 2B, the width dimension of the groove portion 12 being the distance between the two protruding portions 12A, that is, the width dimension of the holding surface 10A is indicated by W. The width dimension of the groove portion 12 (the width dimension of the holding surface 10A) W will be described below. As described below, the hair pulled out from the scalp side is disposed so as to extend in a direction substantially orthogonal to the width direction of the groove portion 12. It can be exemplified that the distance K3 between the upper surface of the protruding portion 12A constituting the groove portion 12 and the holding surface 10A is in the range of 2 mm or more and 6 mm or less.

In the present embodiment, the fixing portion 20 is attached to the holding base 10, and is disposed at a position farther from the end portion 4B of the pedestal portion 4 than the holding surface 10A. The fixing portion 20 is formed by, for example, bending an elongated sheet member covered with release paper on one surface and provided with an adhesive layer into a “dogleg shape” or an “L shape” so that the surface provided with the adhesive layer faces inward. One of two bent and connected outer surfaces of the sheet member is joined to the holding base 10 (see FIG. 1). As the sheet member, paper or a resin sheet can be used. As described below, the release paper is peeled off, the hair is sandwiched between two facing adhesive surfaces, and the adhesive surfaces are bonded to each other, whereby the hair can be fixed to the holding base 10.

The holding base 10 has an upper surface lower in height than the holding surface 10A, and the fixing portion 20 is attached to the upper surface lower than the holding surface 10A. However, the present invention is not limited thereto, and for example, there may also be a case where the fixing portion 20 is attached to the upper surface of the holding base 10 having the same height as the holding surface 10A. Since the thickness of the sheet member and the adhesive layer of the fixing portion 20 is small, it can be said that the fixing portion 20 is disposed at substantially the same height as the holding surface 10A.

In any case, in the hair collection tool 2 according to the present embodiment, since the fixing portion 20 is attached to the upper surface of the holding base 10 at a position farther from the end portion 4B of the pedestal portion 4 than the holding surface 10A, the hair placed on the holding surface 10A can be reliably fixed and held by the holding base 10 having sufficient rigidity.

In addition, a nonslip uneven surface 100 is provided in a region farther from the side surface 10B than the fixing portion 20 of the collecting surface 4A on the upper surface of the pedestal portion 4. Furthermore, the collecting surface 4A of the pedestal portion 4 is provided with a scale for measuring the length of the drawn hair. In the figures, scales of 5 cm and 10 cm are drawn, but the scale is not limited thereto, and scales of any length can be shown according to the application. Furthermore, in a region between the end portion 4B of the pedestal portion 4 on the collecting surface 4A and the side surface 10B of the holding base 10, a dotted line serving as a guideline of a position where the hair is cut with scissors is shown.

### (Method for Collecting Hair)

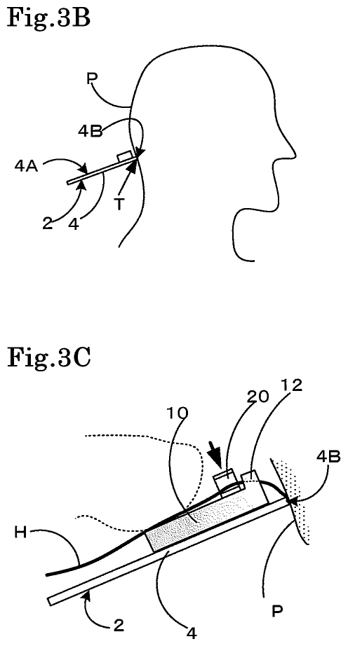

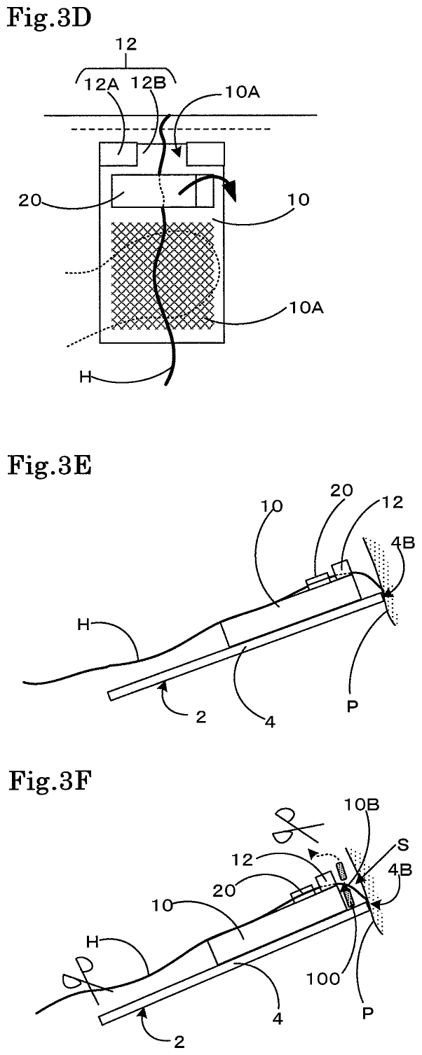

Next, a method for collecting hair using the hair collection tool 2 according to the first embodiment will be described with reference to FIGS. 3A to 3F. FIG. 3A is a diagram schematically showing a position where a hair sample is collected, and a side view showing the back of the head. FIG. 3B is a diagram schematically showing a state where the hair collection tool is applied to the scalp in order to collect a hair sample using the hair collection tool according to the first embodiment of the present invention. FIG. 3C is a side view schematically showing a state where the hair is taken out from the scalp so as to be placed on the holding surface in order to collect a hair sample, from the state in FIG. 3B. FIG. 3D is a plan view of the state shown in FIG. 3C as viewed from the collecting surface side. FIG. 3E is a side view schematically showing a state where the hair taken out is fixed by the adhesive layer of the fixing portion, from the state shown in FIGS. 3C and 3D. FIG. 3F is a side view schematically showing a state of cutting hair by inserting scissor blades into a space surrounded by a side surface, a collecting surface, and a scalp, from the state shown in FIG. 3E. In the figures, only one hair of a plurality of hairs taken out is shown.

FIG. 3A shows a case of collecting hair in a collection region T where a slightly lower side of the most protruding portion X of the back of the head and a longitudinal center line Y of the head intersect. First, the hair is combed left and right along the longitudinal center line Y using a comb or the like. The combed hair around the collection region T is temporarily held with a pin 102 or the like so that the hair is not hung in the collection region T (see FIG. 3A). Next, the release paper covering the adhesive layer of the fixing portion 20 is peeled off. Then, hair of the amount of an area of about 1 cm square (about 1 cm²) in the collection region T is pinched. At this time, it is preferable to evenly pinch the hair in the square. Accordingly, about 50 to 60 hairs suitable for the test can be collected. However, the number of hairs to be collected is not limited to 50 to 60, and an optimum number of hairs can be collected according to the type and use of the test.

Next, the pedestal portion 4 is applied to the scalp P at the hair root of the collection site so as to be at a right angle (see FIG. 3B). At this time, the pedestal portion 4 is applied to the scalp P in such a state where the collecting surface 4A faces upward. Then, in a state where the end portion 4B is pressed against the scalp P with one hand, the hair is taken out from the scalp side with the other hand so as to be placed on the holding surface 10A of the holding base 10 (see FIGS. 3C and 3D).

The hair taken out passes through the holding surface 10A, passes between the two opposing adhesive surfaces of the fixing portion 20, and extends to a region where the uneven surface 100 is provided. The length of the hair to be taken out is preferably 5 cm or more. In addition, when the length of the hair taken out exceeds 10 cm, as described below, the hair can be cut with scissors using the scale shown on the collecting surface 4A and can also have a total length of 10 cm or less.

Then, in a state where the hair taken out is in contact with the holding surface 10A, passes between the two opposing adhesive surfaces of the fixing portion 20, and extends to the nonslip uneven surface 10C, the hair is pressed against the uneven surface 100 with a finger, and the state is held. At this time, the hair is held in a state of not overlapping each other on the holding surface 10A and not slackening. Since the hair collection tool 2 according to the present embodiment has this nonslip uneven surface 100, it is possible to reliably hold the hair taken out.

In the hair collection tool 2 according to the present embodiment, the holding base 10 includes the groove portion 12 having the holding surface 10A as a bottom surface, and the width dimension (that is, the width dimension of the holding surface 10A) W of the groove portion 12 in a direction intersecting with the direction in which the taken out hair extends is determined according to the number of hairs to be collected. As described above, in the scalp P, 50 to 60 hairs can be taken out by taking out hairs in a region of 1 cm square, but this is merely a guideline, and it is also a fact that the density of hairs grown on the scalp varies among individuals. In the present embodiment, a desired number of hairs can be more reliably collected by arranging the hairs to the full width of groove portion 12 (the width of holding surface 10A) so that the hairs do not overlap with each other.

More specifically, for example, when 50 to 60 hairs are arranged so as not to overlap each other, the width dimension in a direction orthogonal to the direction in which the hairs extend is about 5 to 6 mm. Therefore, by setting the width dimension (width dimension of the holding surface 10A) W of the groove portion 12 to 5 to 6 mm, 50 to 60 hairs can be easily collected.

In a test including an accumulation amount of minerals using hair, about 50 to 60 hairs are generally collected, but depending on the content and use of the test, a smaller number of hairs may be collected or a larger number of hairs may be collected.

In consideration of these, it can be said that the width dimension (width dimension of the holding surface 10A) W of the groove portion 12 is preferably in the range of 3 mm or more and 8 mm or less, and more preferably in the range of 5 mm or more and 6 mm or less. Accordingly, by arranging the hairs to the full width of groove portion 12 (the width of holding surface 10A) so as not to overlap each other, it is possible to easily and reliably collect an amount of hairs suitable for the test.

Next, as indicated by arrows in FIGS. 3C and 3D, in a state where the hair passes between the two opposing adhesive surfaces of the fixing portion 20, the sheet member on the upper side of the fixing portion 20 is pressed downward with a finger, and the upper and lower adhesive surfaces are bonded to each other in a state where the hair is sandwiched. Furthermore, the bonded portion is strongly pressed with a finger, and firmly fixed so that the hair is not detached from the fixing portion 20. A state in which the taken out hair is fixed by the adhesive layer of the fixing portion 20 in this manner is shown in FIG. 3E.

Next, as shown in FIG. 3F, the hair from the scalp P to the fixing portion 20 is set in a state of being taut, and scissor blades 100 are inserted into a space S surrounded by the side surface 10B of the holding base 10, the collecting surface 4A of the pedestal portion 4, and the scalp P to cut the hair. At this time, by advancing scissors along the dotted line shown on the collecting surface 4A, hair can be cut straight at an appropriate position.

There are various sizes of scissors, but as long as the use is for cutting hair for collecting a hair sample, medium or small scissors such as those sold as stationery will sufficiently satisfy the function. In addition, even in the case of large scissors, the width of the region near the blade edge is the same as the width of the blade of the medium or small scissors.

The space S surrounded by the side surface 10B, the collecting surface 4A, and the scalp P into which the scissor blades are inserted is defined by the distance K1 between the end portion 4B of the pedestal portion 4 and the side surface 10B of the holding base 10 and the distance K2 from the collecting surface 4A to the holding surface 10A.

If the distance K1 is too short, the distance K1 is narrower than the thickness of the scissor blades 100, and there is a possibility that the scissor blades 100 cannot be inserted. On the other hand, if the distance K1 is too long, the scissor blades 100 are too far from the scalp P, and there is a possibility that the hair is cut in a region far from the scalp P of the hair. In this case, hair near the scalp suitable for the test cannot be collected. In consideration of these, the distance K1 is preferably in a range of 3 mm or more and 8 mm or less, and more preferably in a range of 4 mm or more and 6 mm or less. Accordingly, the scissor blades 100 can be easily inserted into the space P, and the hair near the scalp can be reliably collected.

If the distance K2 is too short, the distance K2 is narrower than the width of the scissor blades 100, and there is a possibility that the scissor blades 100 cannot be inserted. On the other hand, if the distance K2 is too long, the scissor blades 100 are too far from the scalp P, and there is a possibility that the hair is cut in a region far from the scalp P of the hair. In addition, the height of the holding base 10 is too large, attaching of the holding base 10 to the pedestal portion 4 is unstable, and there is a possibility that the holding base 10 is bulky even in transportation. In consideration of these, the distance K2 is preferably in a range of 4 mm or more and 10 mm or less, and more preferably in a range of 5 mm or more and 8 mm or less. Accordingly, the scissor blades 100 can be easily inserted into the space P, the hair near the scalp can be reliably collected, and a compact hair collection tool 2 can be obtained.

If the length of the cut hair is longer than 10 cm and extends so as to protrude from the pedestal portion 4, the hair can be cut with scissors using the scale of 10 cm provided on the collecting surface 4A of the pedestal portion 4, and the length of the hair can also be made 10 cm or less. In the present embodiment, since scales for measuring the length of the hair taken out to the pedestal portion 4 are shown, the length of the collected hairs can be reliably aligned within a desired length.

Then, the hair collection tool 2 to which the collected hair is fixed can be stored, for example, in a resin bag having a sealing function, and the bag can be put in an envelope and sent to an inspection institution by mail. Since the pedestal portion 4 of the hair collection tool 2 has predetermined rigidity, it is possible to prevent the hair sample from being damaged during transportation. It should be noted that when the hair is stored in the bag, the hair is preferably stored so that the scalp side of the hair, that is, the end portion 4B side of the pedestal portion 4 is positioned on the bottom side of the bag. Accordingly, it is possible to prevent the scalp side of the hair important for the test from being damaged during handling such as putting the hair into a bag or taking out the hair from the bag.

### (Hair Collection Tool According to Second Embodiment)

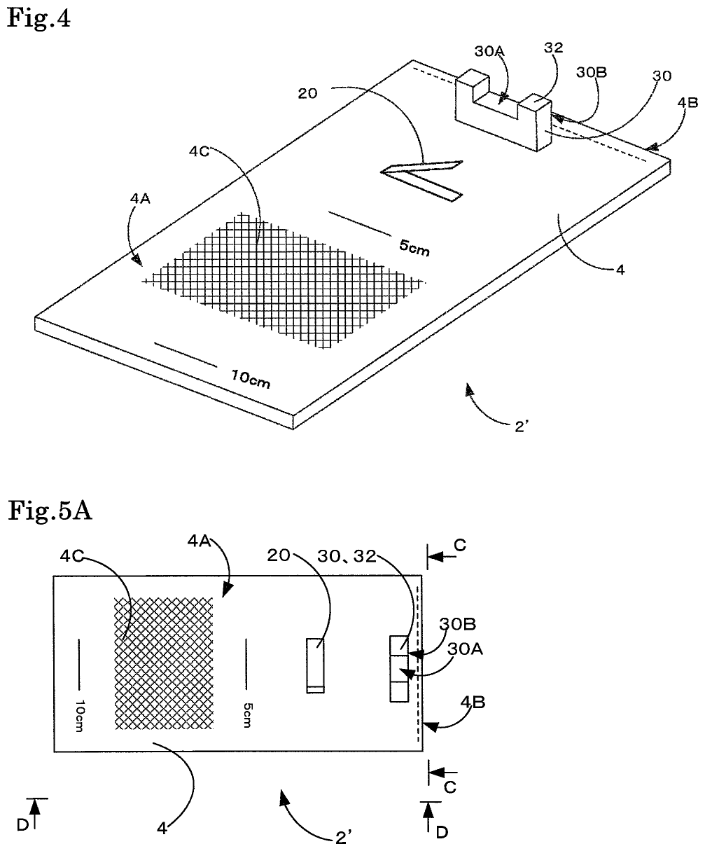

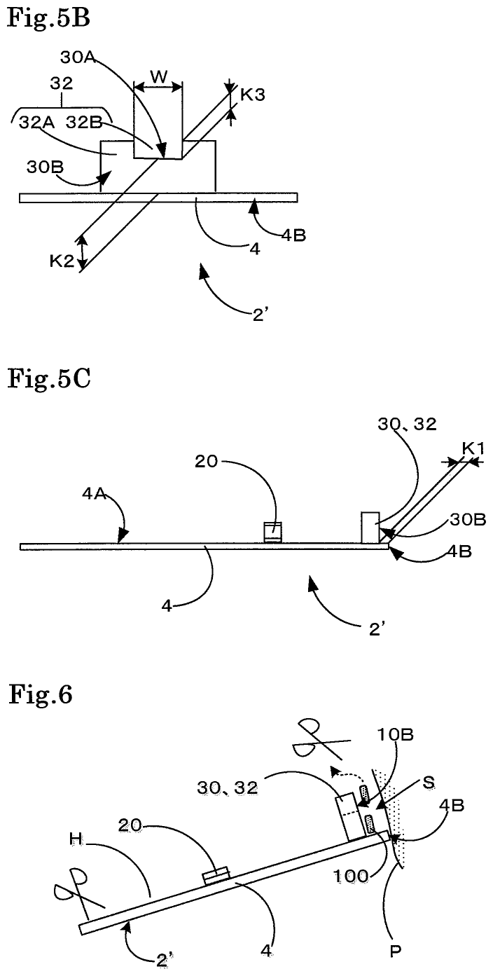

Next, a hair collection tool 2′ according to a second embodiment of the present invention will be described with reference to FIG. 4 and FIGS. 5A to 5C. FIG. 4 is a perspective view schematically showing a hair collection tool according to the second embodiment of the present invention. FIG. 5A is a plan view schematically showing a hair collection tool according to the second embodiment of the present invention. FIG. 5B is a side view showing a view from arrows C-C in FIG. 5A. FIG. 5C is a side view showing a view from arrows D-D in FIG. 5A.

In the first embodiment described above, the fixing portion 20 is attached to the upper surface of the holding base 10, but the present embodiment is different in that the fixing portion 20 is attached to the collecting surface 4A of the pedestal portion 4. Along with this, the holding base 30 according to the present embodiment is different in shape from the holding base 10 according to the first embodiment. The holding base 30 mainly includes only the groove portion 32 in which the recessed portion 32B is arranged between the two protruding portions 32A and a base region thereof.

In the present embodiment, the fixing portion 20 is attached on the collecting surface 4A at a position farther from the end portion 4B of the pedestal portion 4 than the holding surface 30A. As shown in FIG. 5C, the fixing portion 20 is disposed slightly separately from the holding surface 30A and is disposed at a position lower than the holding surface 30A as compared with the case of the first embodiment.

In the present embodiment, since the fixing portion 20 is attached to the collecting surface 4A, the arrangement position of the collecting surface 4A can be relatively freely changed. Furthermore, since the fixing portion 20 is disposed at a position lower than the holding surface 30A, it is possible to easily apply a tensile force to (easily tightly stretch) the taken out hair using the height difference between the holding surface 30A and the fixing portion 20.

Furthermore, a nonslip uneven surface 4C may be provided on the collecting surface 4A. Accordingly, it is possible to reliably hold the taken out hair. The other points are basically similar to those of the first embodiment described above, and thus further description thereof is omitted.

The method of collecting hair using the hair collection tool 2′ according to the second embodiment is substantially similar to the method of collecting hair using the hair collection tool 2′ according to the first embodiment described above. FIG. 6 is a side view schematically showing a state of inserting scissor blades into a space surrounded by the side surface, the collecting surface, and the scalp to cut hair in order to collect a hair sample using a hair collection tool according to the second embodiment of the present invention.

In the hair collection tool 2′ according to the second embodiment, as described above, since the fixing portion 20 is disposed at a position lower than the holding surface 30A, the hair from the root of the hair on the scalp side to the fixing portion 20 is easily taut when the hair is cut. In addition, when the holding surface 30A is disposed high and the fixing portion 20 is disposed low, when the hair is held by being pressed against the uneven surface 4C with a finger in the step corresponding to FIG. 3C, the hair passing between the two opposing adhesive layers of the fixing portion 20 can easily take a clearance from the adhesive layer on the lower side.

### (Hair Collection Tool According to Third Embodiment)

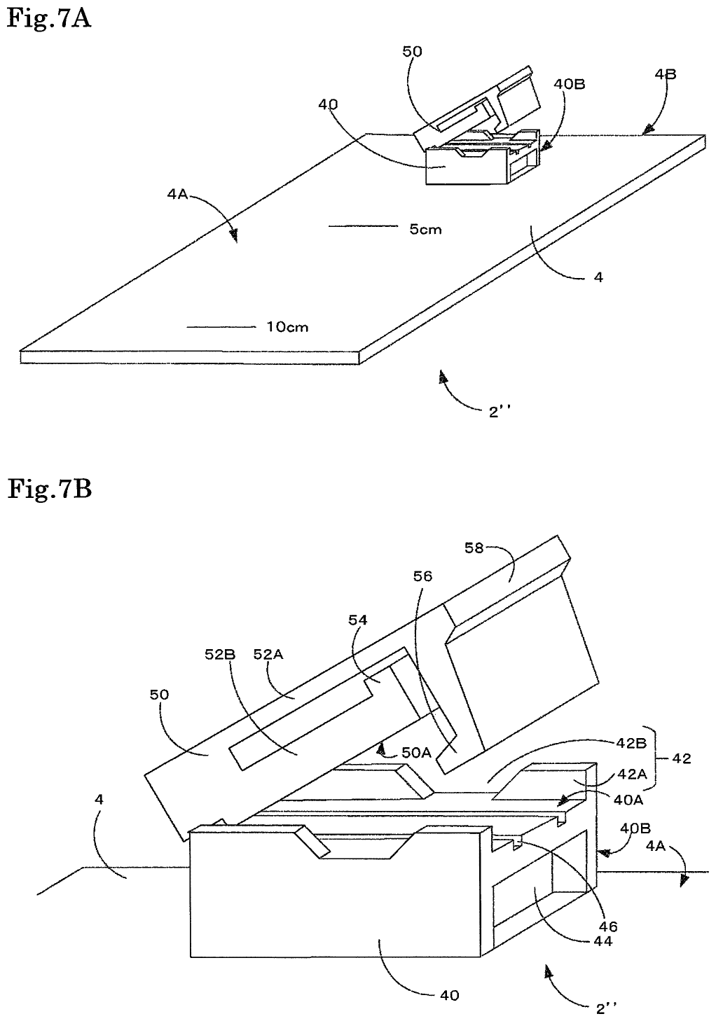

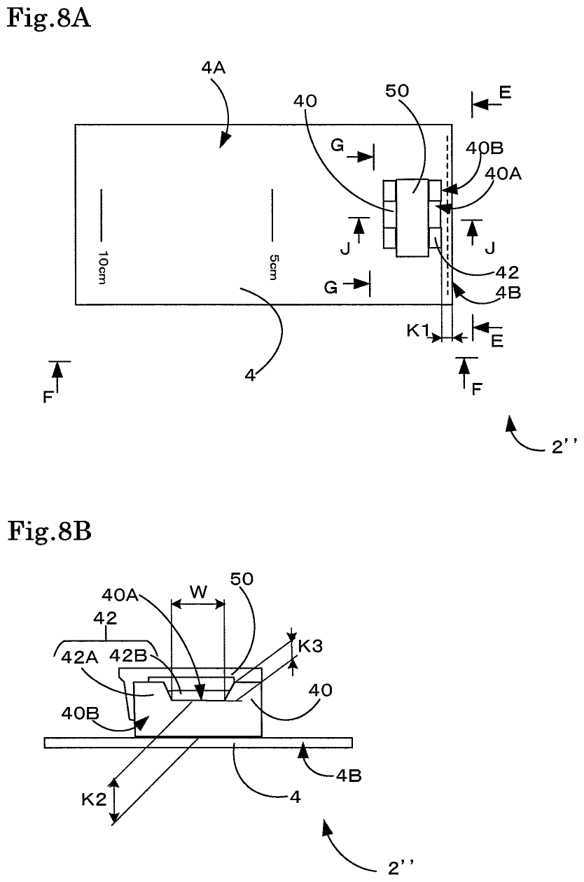

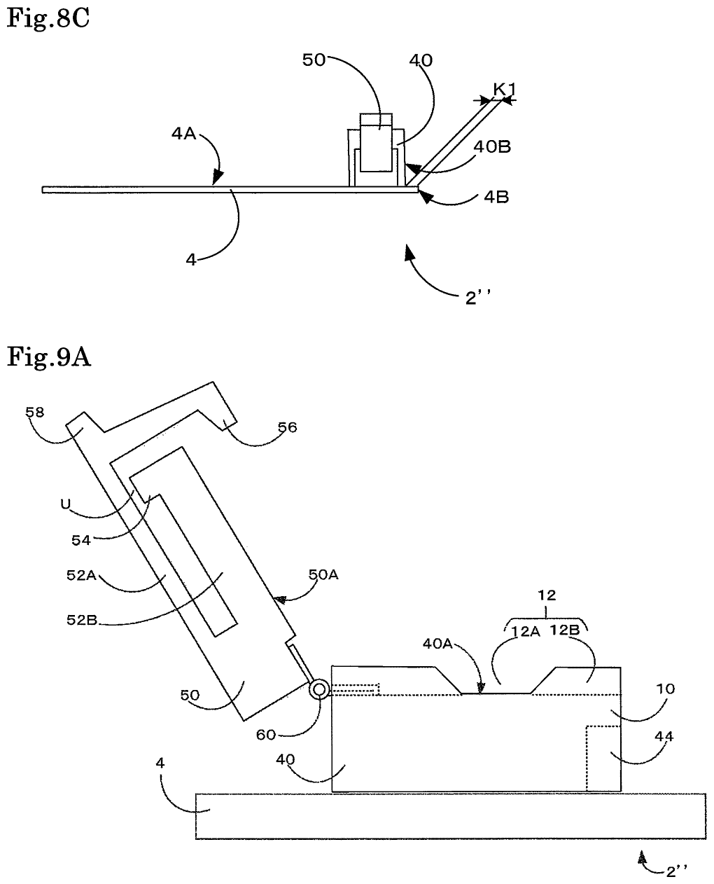

Next, a hair collection tool 2″ according to a third embodiment of the present invention will be described with reference to FIGS. 7A and 7B and FIGS. 8A to 8C. FIG. 7A is a perspective view schematically showing a hair collection tool according to the third embodiment of the present invention. FIG. 7B is a perspective view schematically showing a periphery of a holding base and a fixing portion of the hair collection tool shown in FIG. 7A. FIG. 8A is a plan view schematically showing a hair collection tool according to the third embodiment of the present invention. FIG. 8B is a side view showing a view from arrows E-E in FIG. 8A. FIG. 8C is a side view showing a view from arrows F-F in FIG. 8A.

A hair collection tool 2″ according to the third embodiment of the present invention includes a holding base 40 attached to the pedestal portion 4, and has a structure in which a fixing portion 50 is rotatably attached to the holding base 40 through a rotating shaft. In a state where the fixing portion 50 is opened with respect to the holding base 40, the hair H is placed on the holding surface 40A of the holding base 40, the fixing portion 50 is closed, and the hair H is sandwiched and held between the holding surface 40A of the holding base 40 and the fixing surface 50A of the fixing portion 50.

At this time, the fixing portion 50 and the holding base 40 are fixed in a detachable state by snap-fitting by fitting between the snap arm 56 formed in the fixing portion 50 and the snap groove 44 formed in the holding base 40. Here, the snap-fitting is a mechanical connection method used for coupling members made of an elastic material such as metal or resin, and is a method of fixing by fitting using elasticity of the material. In the present embodiment, both the holding base 40 and the fixing portion 50 are formed of a resin material having elasticity.

As shown in FIGS. 8A to 8C, the present embodiment is also arranged to have distances K1, K2, and K3 similar to those of the first and second embodiments. That is, also in the present embodiment, the distance K1 is longer than the thicknesses of the scissor blades that cut hair, and the distance K2 is longer than the widths of the scissor blades. Specifically, the distance K1 is preferably in a range of 3 mm or more and 8 mm or less, and more preferably in a range of 4 mm or more and 6 mm or less. The distance K2 is preferably in a range of 4 mm or more and 10 mm or less, and more preferably in a range of 5 mm or more and 8 mm or less.

In addition, it can be exemplified that the distance K3 between the upper surface of the protruding portion 42A constituting the groove portion 42 and the holding surface 40A is also in the range of 2 mm or more and 6 mm or less.

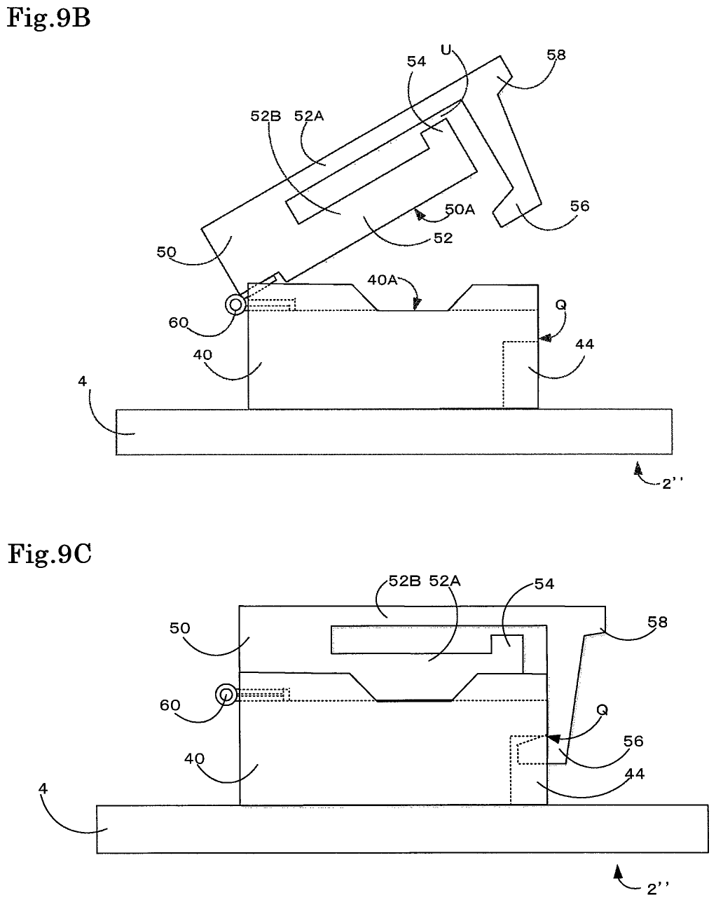

Next, with reference to FIGS. 9A to 9C, the structures of the holding base 40 and the fixing portion 50 will be described in more detail, and the movement of the fixing portion 50 will also be described in more detail. FIGS. 9A to 9C are side views showing views from arrows G-G in FIG. 8A, FIG. 9A is a view showing a state in which the fixing portion is opened, FIG. 9B is a view showing a state in which the fixing portion is further rotated in a closing direction from the state shown in FIG. 9A, and FIG. 9C is a view showing a state in which the fixing portion is closed.

The fixing portion 50 is rotatably attached to the holding base 40 through a rotating shaft 60. In the present embodiment, the fixing portion 50 and the holding base 40 are coupled by a hinge having a rotating shaft 60. However, the present invention is not limited thereto, and any other structure can be adopted as long as the structure has a fixing portion 50 that can be opened and closed around the rotating shaft 60.

The fixing portion 50 includes a base portion 52A and a beam portion 52B connected to the base portion 52A on the rotating shaft 60 side. Furthermore, a support portion 54 is formed at a tip on the opposite side from the rotating shaft 60 of the beam portion 52. In addition, a snap arm 56 is formed at a tip on the opposite side from the rotating shaft 60 of the base portion 52A, and a grip 58 is formed on the upper side of the snap arm 56. On the other hand, a snap groove 44 to be fitted to the snap arm 56 of the fixing portion 50 is formed in the holding base 40.

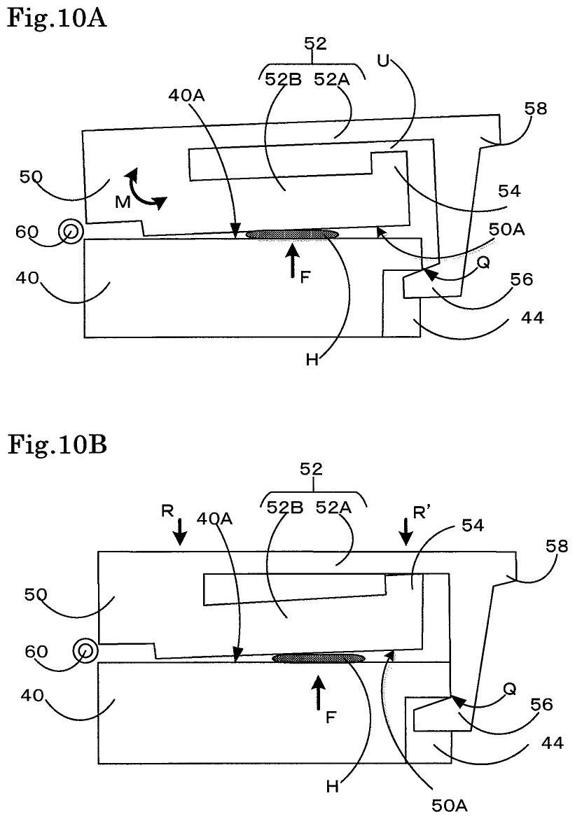

In a state where no load is applied, a gap U is present between the support portion 54 of the beam portion 52B and the base portion 52A. That is, in a state where no load is applied, the base portion 52A and the beam portion 52B have a U-shape or a C-shape, and the beam portion 52B is connected to the base portion 52A in the form of a cantilever beam. As will be described below with reference to FIG. 10B, when the hair H is sandwiched and fixed by the holding base 40 and the fixing portion 50, the resin beam portion 52B is elastically deformed to eliminate the gap U, and the support portion 54 of the beam portion 52B and the base portion 52A are brought into contact with each other.

When the user holds the grip 58 and rotates the fixing portion 50 in the closing direction from the state where the fixing portion 50 is largely opened with respect to the holding base 40 as shown in FIG. 9A, the fixing portion rotates to a position as shown in FIG. 9B. When the fixing portion 50 is further rotated in the closing direction, the snap arm 56 at the tip of the beam portion 52B abuts on the corner portion Q on the upper outer side of the snap groove 44 provided in the holding base 40. Furthermore, when the beam portion 52B is pushed by holding the grip 58, the snap arm 56 is elastically deformed to be in a fixed state in which the snap arm 56 and the snap groove 44 are fitted.

When the fixing portion 50 is rotated in the opening direction with the grip 58 held from this fixed state, the snap arm 56 is elastically deformed, the fitting state of the snap arm 56 and the snap groove 44 is released, and the snap arm 56 is separated from the snap groove 44. Furthermore, by rotating the fixing portion 50 in the opening direction, it is possible to return to the position shown in FIG. 9A through the state shown in FIG. 9B. The fixing portion 50 can be further rotated in the opening direction, and can be rotated by 180 degrees or more with respect to the holding base 40. The grip 58 has a shape very easy to hold, and can easily perform fitting and release fitting on the snap arm 56 and the snap groove 44.

As described above, in the present embodiment, the holding base 40 and the fixing portion 50 are fixed in a detachable state by snap-fitting. Accordingly, the user can easily sandwich the hair H between the holding base 40 and the fixing portion 50, and can easily release the restraint of the hair H.

### <Function of Holding Hair>

Next, the function of holding the hair H with the holding base 40 and the fixing portion 50 will be described in more detail with reference to FIGS. 10A and 10B. FIGS. 10A and 10B are side views showing views from arrows G-G in FIG. 8A, FIG. 10A is a view showing a function of holding hair by the beam portion immediately before the fixing portion is closed, and FIG. 10B is a view showing a function of holding hair by the beam portion in a state where the fixing portion is closed.

When the fixing portion 50 is rotated in the closing direction with the hair H placed on the holding base 40, the snap arm 56 of the fixing portion 50 abuts on the corner portion Q on the upper outer side of the snap groove 44 of the holding base 40. When the fixing portion 50 is further pushed in the closing direction, the snap arm 56 rotates in the closing direction while being elastically deformed. Then, as shown in FIG. 10A, the fixing surface 50A of the fixing portion 50 and the hair H abuts on each other. Accordingly, the hair H is pushed downward by the fixing surface 50A of the fixing portion 50.

At this time, an upward force F is applied to the beam portion 52B of the fixing portion 50 from the holding surface 40A of the holding base 40 and the hair H. Therefore, the beam portion 52B in the form of a cantilever beam made of resin having elasticity receives a bending moment M in a coupling region with the base portion 52A and is bending-deformed (elastically deformed). With the force associated with the bending deformation (elastic deformation), a sufficient downward force can be applied to the hair H. Then, as shown in FIG. 10B, when the snap arm 56 of the fixing portion 50 and the snap groove 44 of the holding base 40 are fitted and in a fixed state, the gap U between the support portion 54 of the beam portion 52B and the base portion 52A disappears, and the support portion 54 is in a state of abutting on the base portion 52A.

Accordingly, the beam portion 52B is in the form of a double-supported beam, and the upward force F applied from the holding surface 40A of the holding base 40 and the hair H is supported by the reaction forces R and R′ on both sides. The beam portion 52B in the form of a double-supported beam having elasticity is bending-deformed (elastically deformed) by this force, but a sufficient downward force can be applied to the hair H by the force accompanying this bending deformation (elastic deformation). Accordingly, it is possible to firmly hold the hair H with the holding surface 40A of the holding base 40.

As described above, in the present embodiment, the fixing surface 50A in contact with the hair H is formed in the beam portion 52B having elasticity of the fixing portion 50, and a force accompanying bending deformation of the beam portion 52B is applied to the hair H.

Accordingly, since the hair H can be held by the force accompanying the bending deformation of the beam portion 52B having elasticity of the fixing portion 50, even if an impact or the like is applied to the hair collection tool 2″, the bending-deformed beam portion 52B follows the rapid fluctuation and can always hold the hair H in a stable state.

### <Functions of Holding Protruding Portion and Holding Recessed Portion>

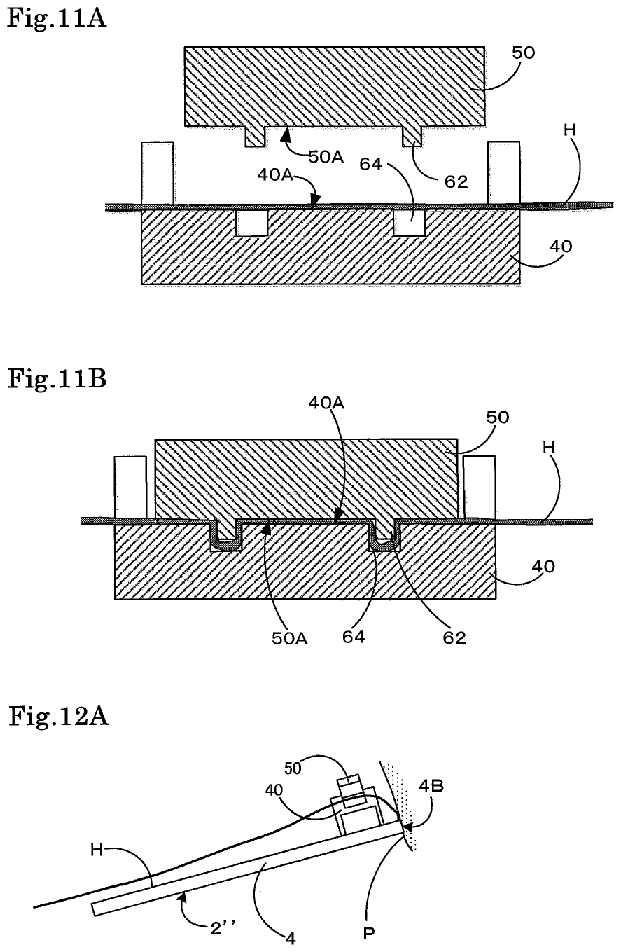

Next, the functions of the holding protruding portion that holds hair and the holding recessed portion will be described with reference to FIGS. 11A and 11B. FIGS. 11A and 11B are side views showing views from arrows J-J in FIG. 8A and are views showing functions of the holding protruding portion and the holding recessed portion, FIG. 11A is a view showing a state in which the fixing portion is opened, and FIG. 11B is a view showing a state in which the fixing portion is closed.

In the present embodiment, the holding protruding portion 62 is formed on the fixing surface 50A of the fixing portion 50, and the holding recessed portion 64 is formed on the holding surface 40A of the holding base 40. However, the present invention is not limited thereto, and the unevenness may be reversed between the fixing surface 50A and the holding surface 50A. The holding protruding portion 62 and the holding recessed portion 64 extend in a direction intersecting with the direction in which the hair H to be held extends, and are formed at two places in the direction in which the hair H extends. The holding protruding portion 62 and the holding recessed portion 64 may be orthogonal to the direction in which the hair H to be held extends, or may extend in a direction oblique to the direction in which the hair H to be held extends. The holding protruding portion 62 and the holding recessed portion 64 are preferably formed from one end portion to the other end portion in a direction intersecting the direction in which the hair H extends on the fixing surface 50A and the holding surface 40A.

The holding protruding portion 62 and the holding recessed portion 64 are arranged at positions to be fitted to each other at a rotational position where the fixing portion 50 is closed with respect to the holding base 40. More specifically, the dimension of the holding protruding portion 62 is formed to be slightly smaller than the dimension of the holding recessed portion 64, and the dimension of the holding protruding portion 62 is inserted into the holding recessed portion 64 through a slight gap.

As shown in FIG. 11A, the hair H is placed on the holding surface 40A of the holding base 40 at the rotational position where the fixing portion 50 is opened. In this state, the fixing portion 50 is rotated in the closing direction, and the fixing portion 50 and the holding base 40 are fixed by the fitting between the snap arm 56 and the snap groove 44. This state is shown in FIG. 11B. At this time, the holding protruding portion 62 is inserted into the holding recessed portion 64, and the hair H is curved along the inserted holding protruding portion 62 and enters the holding recessed portion 64.

Accordingly, since the hair H is sandwiched between the holding protruding portion 62 and the holding recessed portion 64 that are to be fitted to each other, the hair H is more firmly fixed between the fixing portion 50 and the holding base 40.

As described above, in the present embodiment, the protruding portion 62 is formed on one of the holding surface 40A of the holding base 40 and the fixing surface 50A of the fixing portion 50, and the recessed portion 64 is formed on the other, and in a state where the hair H is sandwiched between the holding surface 40A and the fixing surface 50A, the hair H is sandwiched and held between the protruding portion 62 and the recessed portion 64 fitted to each other. Accordingly, the hair H can be more firmly fixed between the fixing portion 50 and the holding base 40.

### <Method for Collecting Hair>

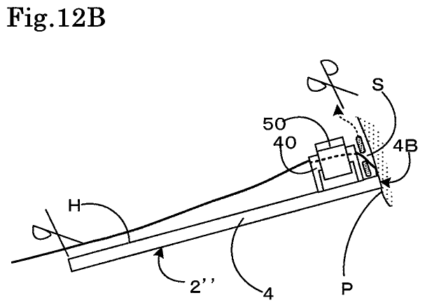

Next, a method for collecting hair sample using the hair collection tool 2″ according to the third embodiment of the present invention will be described with reference to FIGS. 12A and 12B. FIG. 12A is a side view schematically showing a state where in order to collect a hair sample using the hair collection tool according to the third embodiment of the present invention, the hair collection tool is applied to the scalp, and the hair is taken out from the scalp so as to be placed on a holding surface of a holding base. FIG. 12B is a side view schematically showing a state where the hair taken out is sandwiched and fixed by the holding surface of the holding base and the fixing surface of the fixing portion, and scissor blades are inserted into a space surrounded by the side surface, the collecting surface, and the scalp to cut the hair, from the state shown in FIG. 12A.

In the hair collection tool 2″ according to the third embodiment, the structure of the fixing portion 50 is different from that of the above embodiments, but the method for collecting a hair sample is basically similar to that of the above embodiments. As shown in FIG. 12A, in a state where the end portion 4B of the hair collection tool 2″ is applied to the scalp P, a predetermined amount of hair H is taken out from the scalp P side so as to be placed on the holding surface 40A of the holding base 40. Then, the fixing portion 50 is rotated in a closing direction in a state where the hair H is taut, and the fixing portion 50 and the holding base 40 are fixed by snap-fitting. Accordingly, the hair H is held in a state of being sandwiched between the holding base 40 and the fixing portion 50.

Then, as shown in FIG. 12B, the hair from the scalp P to the holding base 40 and the fixing portion 50 is set in a state of being taut, and scissor blades 100 are inserted into a space S surrounded by the side surface 40B of the holding base 40, the collecting surface 4A of the pedestal portion 4, and the scalp P to cut the hair.

As described above, in the hair collection tool 2″ according to the third embodiment of the present invention, the fixing portion 50 is rotatably attached to the holding base 40 through the rotating shaft 60, and the holding base 40 and the fixing portion 50 are fixed at the rotational position of the fixing portion 50 with the hair H sandwiched between the holding surface 40A of the holding base 40 and the fixing surface 50A of the fixing portion 50.

Accordingly, at the rotational position where the fixing portion 50 is opened with respect to the holding base 40, the hair H is disposed between the two, and the fixing portion 50 is rotated in the closing direction so that the hair H is set to a state of being sandwiched between the holding surface 40A of the holding base 40 and the fixing surface 50A of the fixing portion 50, whereby the two can be fixed. Therefore, the hair H can be reliably held between the holding base 40 and the fixing portion 50 by an easy operation.

### (General)

As described above, a hair collection tool 2, 2′, 2″ according to the first to third embodiments of the present invention includes: a pedestal portion 4 having a thin plate shape and having a collecting surface 4A; a holding base 10, 30, 40 attached to the collecting surface 4A; and a fixing portion 20, 50 attached to the collecting surface 4A through the holding base 10, 40 or directly. The holding base 10, 30, 40 includes a side surface 10B, 30B, 40B erected at a position away from an end portion 4B of the pedestal portion 4 by a distance K1, and a holding surface 10A, 30A, 40A extending along the collecting surface 4A in a direction away from the end portion 4B from a position by a distance K2 above the collecting surface 4A of the side surface 10B, 30B, 40B. The distance K1 is longer than a thickness of scissor blades 100 that cut hair, and the distance K2 is longer than a width of the scissor blades 100. In a state of the end portion 4B being applied to a scalp P, a predetermined amount of hair is taken out from a scalp side so that the predetermined amount of hair is placed on the holding surface 10A, 30A, 40A, hair taken out is fixed by the fixing portion 20, 50, scissor blades 100 are inserted into a space P surrounded by the side surface 10B, 30B, 40B, the collecting surface 4A, and a scalp P to cut hair, and a hair sample is collected.

Since the hair collection tool 2, 2′, 2″ has the distances K1 and K2 longer than the thickness and width of the scissor blades 100, the scissor blades 100 can be easily inserted into the space P surrounded by the side surface 10B, 30B of the holding base 10, the collecting surface 4A of the pedestal portion 4, and the scalp P, and the hair can be reliably cut. Accordingly, a region near the root of the hair useful for analysis can be easily collected. In addition, since the collected hair is fixed to the pedestal portion 4 by the fixing portion 20, 50, the collected hair is protected by the pedestal portion 4 and can be prevented from being damaged during mail or the like. Accordingly, anyone can easily collect a hair sample suitable for analysis, and the collected hair sample can be sent to an inspection institution without being damaged.

Although the embodiments and aspects of the present invention are described, the disclosed content may vary in the details of the configuration, and combinations of elements or changes in order of elements in the embodiments and aspects may be achieved without departing from the scope and spirit of the present invention claimed.

### REFERENCE NUMERAL LIST

2, 2′, 2″ hair collection tool
4 pedestal portion
4A collecting surface
4B end portion
4C uneven surface
10 holding base
10A holding surface
10B side surface
100 uneven surface
12 groove portion
12A protruding portion
12B recessed portion
20 fixing portion
30 holding base
30A holding surface
30B side surface
32 groove portion
32A protruding portion
32B recessed portion
40 holding base
40A holding surface
40B side surface
42 groove portion
42A protruding portion
42B recessed portion
44 snap groove
50 fixing portion
50A fixing surface
52A base portion
52B beam portion
54 support portion
56 snap arm
58 grip
60 rotating shaft
62 holding protruding portion
64 holding recessed portion
100 scissor blade
102 pin
H hair
T collection region
K1, K2, K3 distance
X center line
Y most protruding portion
S space
U gap
Q corner portion
F force
R, R′ reaction force

1. A hair collection tool comprising:
a pedestal portion having a thin plate shape and having a collecting surface;
a holding base attached to the collecting surface; and
a fixing portion attached to the collecting surface through the holding base or directly,
wherein the holding base includes a side surface erected at a position away from an end portion of the pedestal portion by a first distance, and a holding surface extending along the collecting surface in a direction away from the end portion from a position by a second distance above the collecting surface of the side surface,
wherein the first distance is longer than a thickness of scissor blades that cut hair, and the second distance is longer than a width of the scissor blades, and
wherein in a state of the end portion being applied to a scalp, a predetermined amount of hair is taken out from a scalp side so that the predetermined amount of hair is placed on the holding surface, hair taken out is fixed by the fixing portion, scissor blades are inserted into a space surrounded by the side surface, the collecting surface, and a scalp to cut hair, and a hair sample is collected.

2. The hair collection tool according to claim 1, wherein the first distance is in a range of 3 mm or more and 8 mm or less, and the second distance is in a range of 4 mm or more and 10 mm or less.

3. The hair collection tool according to claim 1, wherein the holding base includes a groove portion having the holding surface as a bottom surface, and a width dimension of the groove portion in a direction intersecting a direction in which hair taken out extends is determined according to the number of hairs to be collected.

4. The hair collection tool according to claim 3, wherein the width dimension of the groove portion is in a range of 3 mm or more and 8 mm or less.

5. The hair collection tool according to claim 1,
wherein the fixing portion is attached to the holding base in a manner configured to be rotated through a rotating shaft, and
wherein at a rotational position of the fixing portion with hair sandwiched between the holding surface of the holding base and a fixing surface of the fixing portion, the holding base and the fixing portion are fixed.

6. The hair collection tool according to claim 5, wherein the holding base and the fixing portion are fixed in a state configured to be detached by snap-fitting.

7. The hair collection tool according to claim 5, wherein the fixing surface in contact with hair is formed in a beam portion having elasticity of the fixing portion, and a force accompanying bending deformation of the beam portion is applied to hair.

8. The hair collection tool according to claim 5, wherein a protruding portion is formed on one of the holding surface of the holding base and the fixing surface of the fixing portion, a recessed portion is formed on another, and in a state where hair is sandwiched between the holding surface and the fixing surface, hair is sandwiched and held between the protruding portion and the recessed portion that are fitted to each other.

9. The hair collection tool according to claim 1, wherein a scale for measuring a length of hair taken out is indicated on the pedestal portion.
